(19) United States
(12) Patent Application Publication
(10) Pub. No.: US 2015/0148656 A1
(43) Pub. Date: May 28, 2015

(54) INFORMATION OBTAINING APPARATUS AND METHOD FOR OBTAINING INFORMATION

(71) Applicant: CANON KABUSHIKI KAISHA, Tokyo (JP)

(72) Inventor: Sayuri Yamaguchi, Tokyo (JP)

(21) Appl. No.: 14/549,392

(22) Filed: Nov. 20, 2014

(30) Foreign Application Priority Data
Nov. 22, 2013 (JP) 2013-242004
Oct. 15, 2014 (JP) 2014-211197

(51) Int. Cl. A61B 5/05

(52) U.S. Cl. CPC A61B 5/0507
USPC 600/407

(57) ABSTRACT

An information obtaining apparatus that obtains information regarding a subject includes a radiation unit configured to focus a terahertz wave having a plurality of different frequencies or a plurality of different beam diameters and radiate the terahertz wave onto the subject, a detection unit configured to detect the terahertz wave that has penetrated the subject or that has been reflected by the subject, a spectrum obtaining unit configured to obtain a spectrum of optical characteristics of the subject using a result of the detection performed by the detection unit, and a determination unit configured to determine a state of the subject on the basis of the spectrum obtained by the spectrum obtaining unit. The determination unit determines the state of the subject on the basis of a difference between a plurality of spectra obtained by the spectrum obtaining unit at a plurality of different positions in the subject.

### BACKGROUND OF THE INVENTION

1. Field of the Invention

The present invention relates to an information obtaining apparatus that obtains information regarding a subject using a terahertz wave and a method for obtaining information.

2. Description of the Related Art

A terahertz wave is an electromagnetic wave including a component in any frequency band within a range of 30 GHz to 30 THz. These years, nondestructive sensing techniques that use terahertz waves are being developed. As application fields of electromagnetic waves in this frequency band, a technique for performing imaging using a fluoroscopic apparatus, a spectroscopic technique for investigating physical properties such as molecular binding by obtaining an absorption spectrum or a complex permittivity of the inside of a substance, and the like have been developed. As applications of such techniques, a medical field in which the non-invasiveness of terahertz waves is utilized is attracting attention.

In Japanese Patent Laid-Open No. 2006-90863, a method for obtaining the spatial distribution of tissue component concentrations unique to each subject by observing a terahertz wave radiated onto a biological tissue is disclosed. In this method, a frequency spectrum of a portion of a target subject whose state is known is obtained in advance to create a database. This database is referred to when the spatial distribution of the tissue component concentrations is obtained.

Thus, in a determination of the state of a biological tissue using a terahertz wave, a database obtained in advance is usually referred to. This is not limited to a terahertz region, but holds true in a determination of a state using a spectrum obtained through infrared spectroscopy or Raman spectroscopy.

In the terahertz region of a frequency spectrum of a biological tissue, however, characteristic peak are particularly few, and a difference in spectra between a normal tissue portion and an abnormal tissue portion is small. In addition, it is known that there is individual variability in the shape of a spectrum and the value of optical characteristics. Therefore, a method for determining a state without referring to a database is desired.

In Japanese Patent Laid-Open No. 2006-90863, a database created using an average value of a plurality of subjects is not used, but a terahertz spectrum of a portion of a target subject whose state is known is used as a database. Therefore, the determination is not affected by individual variability. Since a portion whose state is known needs to be identified and a terahertz spectrum thereof needs to be obtained in advance as a database, however, such a method might not be used for a subject whose state is not known at all.

### SUMMARY OF THE INVENTION

An information obtaining apparatus according to an aspect of the present invention is an information obtaining apparatus that obtains information regarding a subject. The information obtaining apparatus includes a radiation unit configured to focus a terahertz wave having a plurality of different frequencies or a plurality of different beam diameters and radiate the terahertz wave onto the subject, a detection unit configured to detect the terahertz wave that has penetrated the subject or that has been reflected by the subject, a spectrum obtaining unit configured to obtain a spectrum of optical characteristics of the subject using a result of the detection performed by the detection unit, and a determination unit configured to determine a state of the subject on the basis of the spectrum obtained by the spectrum obtaining unit. The determination unit determines the state of the subject on the basis of a difference between a plurality of spectra obtained by the spectrum obtaining unit at a plurality of different positions in the subject.

Further aspects of the present invention will become apparent from the following description of exemplary embodiments with reference to the attached drawings.

### BRIEF DESCRIPTION OF THE DRAWINGS

FIG. 1 is a diagram illustrating the overall configuration of an information obtaining apparatus according to a first embodiment.

FIG. 2 is a flowchart illustrating a method for obtaining information according to the first embodiment.

FIG. 3A illustrates reflectance spectra obtained from a normal tissue portion of a subject in the first embodiment.

FIG. 3B illustrates reflectance spectra obtained from an abnormal tissue portion of the subject in the first embodiment.

FIG. 3C is a histogram illustrating differences between the plurality of spectra obtained from the normal tissue portion and the abnormal tissue portion of the subject at each frequency in the first embodiment.

FIG. 4A is a graph illustrating the frequency dependence of the beam diameter of a terahertz wave in the information obtaining apparatus according to the first embodiment.

FIG. 4B is a schematic diagram illustrating a terahertz beam radiated onto the normal tissue portion in the first embodiment.

FIG. 4C is a schematic diagram illustrating a terahertz beam radiated onto the abnormal tissue portion in the first embodiment.

FIG. 5A includes graphs illustrating relationships between the percentage contents of components of the normal tissue portion of a biological tissue as the subject and the beam diameter in the first embodiment.

FIG. 5B includes graphs illustrating relationships between the percentage contents of components of the abnormal tissue portion of the biological tissue as the subject and the beam diameter in the first embodiment.

FIG. 5C illustrates the reflectance spectra of the components of the biological tissue as the subject in the first embodiment.

FIG. 6 is a hematoxylin and eosin (HE)-stained image of the subject in the first embodiment.

FIG. 7 is a histogram illustrating an example of indices indicating differences between the plurality of spectra in the first embodiment.

FIG. 8A is a diagram illustrating a ceramic porous body in which an area occupied by pores is large as another subject in the first embodiment.

FIG. 8B is a diagram illustrating a ceramic porous body in which the area occupied by pores is small as another subject in the first embodiment.

FIG. 9 is a diagram illustrating the configurations of a generation unit and a detection unit of an image obtaining apparatus according to a second embodiment.

FIG. 10A is a histogram illustrating differences between a plurality of spectra in a first example.

FIG. 10B is a histogram illustrating differences between a plurality of spectra obtained from a subject extracted from another individual in the first example.

FIG. 10C is a histogram illustrating differences between a plurality of spectra obtained from a subject extracted from another individual in the first example.

FIG. 11A illustrates reflectance spectra obtained from a normal tissue portion of a subject in a second example.

FIG. 11B illustrates reflectance spectra obtained from an abnormal tissue portion of the subject in the second example.

FIG. 11C is a histogram illustrating differences between a plurality of spectra obtained from the normal tissue portion and the abnormal tissue portion of the subject at each frequency in the second example.

FIG. 12A illustrates an HE-stained image of the normal tissue portion of the subject in the second example.

FIG. 12B illustrates an HE-stained image of the abnormal tissue portion of the subject in the second example.

FIG. 13 is a histogram illustrating differences between a plurality of spectra in the second example.

### DESCRIPTION OF THE EMBODIMENTS

### First Embodiment

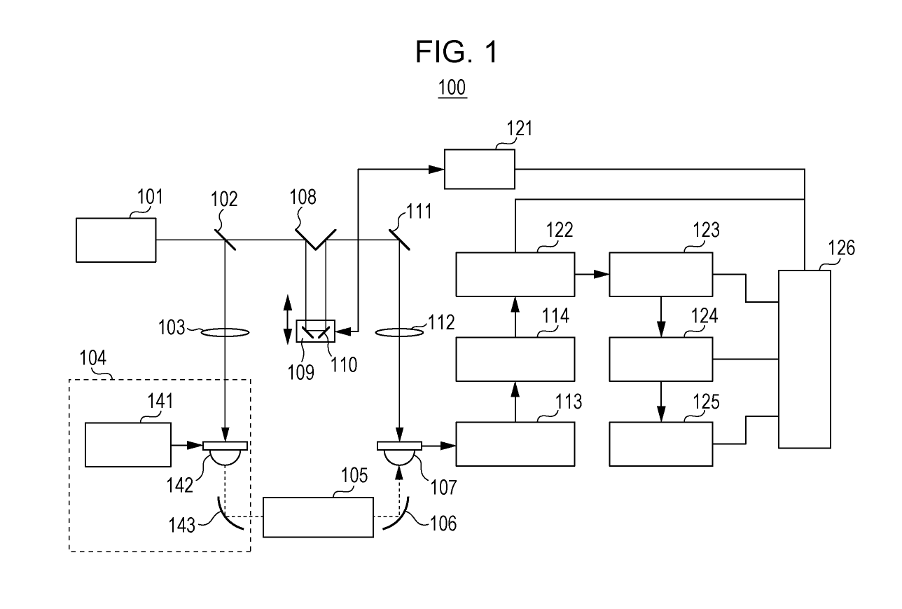

An information obtaining apparatus 100 (hereinafter referred to as an “apparatus 100”) according to a first embodiment will be described with reference to FIG. 1. In this embodiment, a terahertz time-domain spectroscopy (THz-TDS) apparatus is used as the apparatus 100.

First, the configuration of the apparatus 100 will be described with reference to FIG. 1. FIG. 1 is a diagram illustrating the overall configuration of the apparatus 100. The apparatus 100 includes a light source 101, a radiation unit 104, a sample unit 105, a detection unit 107, a control unit 121, a time waveform obtaining unit 122 (hereinafter referred to as an “obtaining unit 122”), a spectrum obtaining unit 123 (hereinafter referred to as an “obtaining unit 123”), a determination unit 124, and an image forming unit 125. The radiation unit 104 includes a bias power supply 141, a generation section 142, and a parabolic mirror 143.

The light source 101 is a component that outputs ultrashort light pulses. The ultrashort light pulses refer to light pulses having a pulse width on the order of femtoseconds. The light source 101 in this embodiment outputs femtosecond laser light (hereinafter referred to as “laser light”) having a pulse width of 10 to 100 femtoseconds. The laser light output from the light source 101 is divided by a one-way mirror 102, and one of obtained laser light beams is focused by a lens 103 and incident on the generation section 142.

The generation section 142 is a component that generates a pulsed terahertz wave (hereinafter also referred to as a “pulsed wave”) upon receiving the laser light. In this embodiment, the generation section 142 is a photoconductive device obtained by forming an antenna on a semiconductor film using a conductor. Alternatively, for example, a generation device in which laser light is radiated onto a surface of a semiconductor substrate or an organic crystal or a generation device in which laser light is guided to a nonlinear crystal may be used. It is only necessary that the generation section 142 generate a pulsed wave upon receiving laser light. A known technique capable of achieving this goal may be applied.

The pulsed wave generated by the generation section 142 is a broadband terahertz wave including a plurality of different frequency components. If such a pulsed wave is focused and radiated onto a subject, the beam diameter of the terahertz wave varies depending on the frequency because diffraction limits are different between frequencies. That is, the pulsed wave is a terahertz wave including a plurality of frequency components having different beam diameters.

The “beam diameter” herein refers to, if a terahertz wave is focused and radiated onto the subject, the beam diameter of the terahertz wave radiated onto a surface of the subject whose information is to be obtained or whose state is to be determined. That is, if the apparatus 100 is configured such that the terahertz wave is focused most sharply on the surface of the subject whose information is to be obtained or whose state is to be determined, the beam diameter refers to a beam diameter at a time when the terahertz wave is focused most sharply.

The bias voltage of the generation section 142 is modulated by the bias power supply 141, and the modulated pulsed wave is radiated by the parabolic mirror 143 onto the subject in the sample unit 105. Thereafter, the pulsed wave that has penetrated the subject or that has been reflected by the subject is radiated by the detection system 106 onto the terahertz wave detection unit 107.

The sample unit 105 is a component in which a sample including a biological tissue as the subject is disposed. In this configuration, the pulsed wave from the generation section 142 is radiated onto the subject by a combination of parabolic mirrors and the like, which are not illustrated, and the pulsed wave that has penetrated the subject or that has been reflected by the subject is incident on the detection unit 107. The subject is disposed on a movable stage with which a position at which the pulsed wave is radiated can be selected. By arranging the subject on the movable stage, the position (measurement point) in the subject at which the pulsed wave is radiated can be changed as necessary.

The apparatus 100 is desirably configured such that even if the position at which the pulsed wave is radiated is changed, the angle and the vertical position of a surface relative to the pulsed wave remain the same. This is because if a photoconductive device is used as the detection unit 107, differences in the angle of the subject and the position of the subject relative to a reference plane affect a resultant spectrum in accordance with the frequency. In a method for determining the state of the subject on the basis of differences between spectra, which will be described later, the frequency dependence of the spectra is focused upon, and accordingly variation in the spectra due to the performance of the apparatus 100 and the shape of the sample is desirably suppressed as much as possible.

The other of the laser light beams obtained as a result of the division by the one-way mirror 102 is subjected to delay control realized by a pair of fixed mirrors 108 and a pair of mirrors 110 mounted on a movable stage 109, and then radiated onto the detection unit 107 through a mirror 111 and a lens 112. The movable stage 109 on which the pair of mirrors 110 is mounted is an adjustment unit that adjusts a timing at which the terahertz pulsed wave is detected by the detection unit 107. More specifically, the movable stage 109 changes the optical path length of the laser light input to the detection unit 107 relative to that of the laser light input to the generation section 142.

In this embodiment, the pair of mirrors 110 and the movable stage 109 are provided between the light source 101 and the detection unit 107 to adjust the optical path length of the laser light from the light source 101 to the detection unit 107. Alternatively, the pair of mirrors 110 and the movable stage 109 may be provided along the path of the laser light input to the generation section 142 to change the optical path length of the laser light input to the generation section 142. In addition, as another method for adjusting the optical path length, for example, a method for changing the optical path length by changing the refractive index in a propagation path may be used.

The detection unit 107 is a component that detects a terahertz wave on the basis of an input pulsed wave and laser light. More specifically, the detection unit 107 detects the instantaneous value of the field strength of the pulsed wave at a moment when the laser light reaches the detection unit 107. In this embodiment, a photoconductive device is used as the detection unit 107. It is only necessary that the detection unit 107 be capable of detecting a pulsed wave using laser light. A known technique capable of achieving this goal may be applied.

A signal obtained from a terahertz pulsed wave, which is a result of the detection performed by the detection unit 107, is transmitted to the obtaining unit 122, the obtaining unit 123, the determination unit 124, and the image forming unit 125 through an amplifier 113 and a lock-in amplifier 114 and converted into information such as image information.

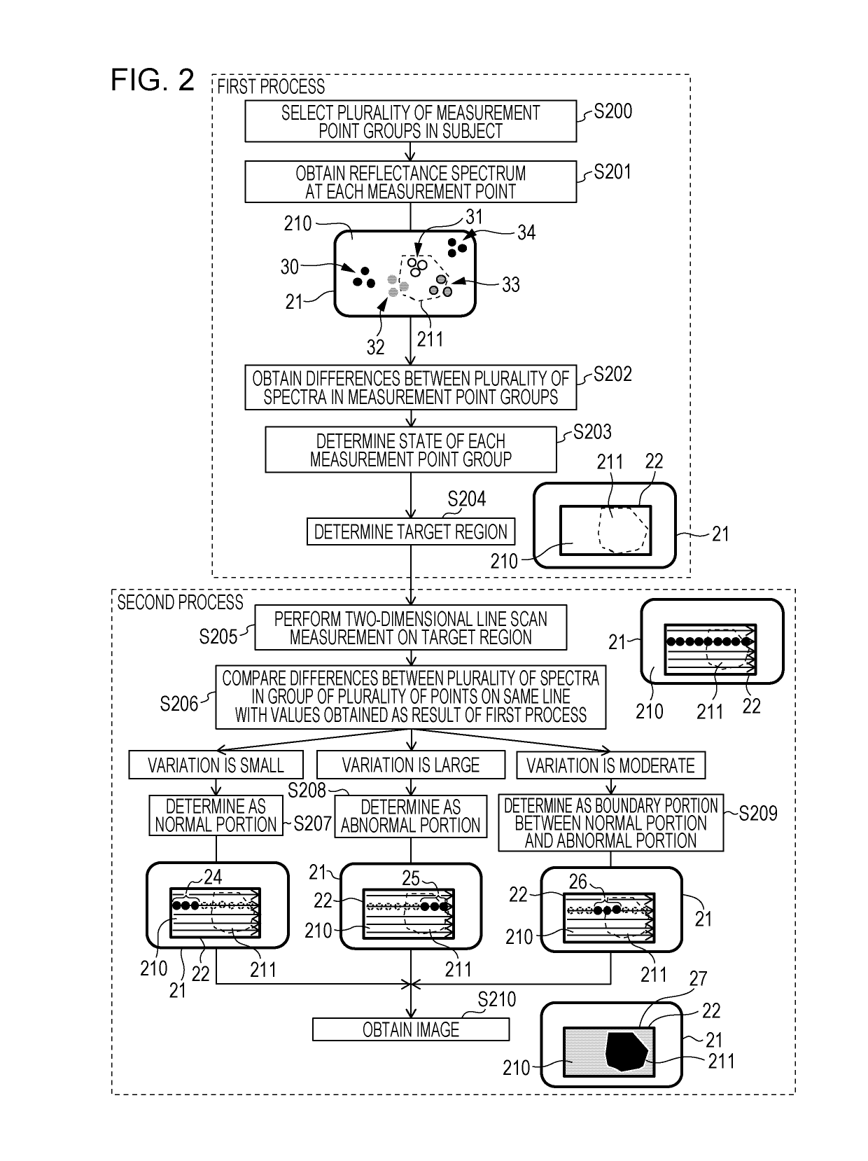

The apparatus 100 includes a computer including a central processing unit (CPU), a memory, and a storage device, and the computer has the functions of the control unit 121, the obtaining unit 122, the obtaining unit 123, the determination unit 124, the image forming unit 125, and the like. The computer also includes a storage unit 126 and stores results of detection performed by the detection unit 107, the time waveforms of terahertz waves, and the like. The storage unit 126 also stores programs corresponding to the steps illustrated in a flowchart of FIG. 2, and each step of processing is performed by the CPU, which reads each of the programs. Each step illustrated in FIG. 2 will be described later.

The control unit 121 is a component that controls the components of the apparatus 100 and mainly controls driving of the movable stage 109 and the stage of the sample unit 105.

The obtaining unit 122 is a component that obtains the time waveform of a terahertz wave. The obtaining unit 122 obtains a time waveform using the optical path length adjusted by an optical delay unit including the movable stage 109 and the pair of mirrors 110 and a result of detection performed by the detection unit 107.

The obtaining unit 123 is a component that obtains the optical characteristics of the subject using the time waveform obtained by the obtaining unit 122 to obtain the spectrum of the optical characteristics drawn along a horizontal axis representing frequency. The obtained spectrum is transmitted to the determination unit 124. In this embodiment, a reflectance spectrum drawn along a horizontal axis representing frequency is used as the spectrum of the optical characteristics. The “spectrum of optical characteristics” in this embodiment, however, is not limited to this, and is defined as a concept including a spectrum drawn along a horizontal axis representing frequency, wavelength, or beam diameter. The optical characteristics herein are defined as a concept including the complex amplitude reflectance, the complex refractive index, the complex permittivity, the reflectance, the refractive index, the absorption coefficient, the permittivity, and the electrical conductivity of the subject.

The determination unit 124 is a component that obtains information regarding the subject by analyzing the time waveform and the reflectance spectrum. More specifically, the determination unit 124 determines the state of the subject at each radiation position. For example, if the subject is a biological tissue, the determination unit 124 distinguishes a normal tissue portion, in which abnormality is not found in the biological tissue, and an abnormal tissue portion, in which abnormality such as cancer is found in cells.

In the apparatus 100, a broadband terahertz wave including a plurality of different frequency components is focused and radiated onto the subject. At this time, since the diffraction limits are different between frequencies, various beam diameters are included in the focused terahertz wave. The state of the subject is determined using changes in the optical characteristics depending on the beam diameter that differs depending on the frequency. The changes in the optical characteristics depending on the beam diameter that differs depending on the frequency are indicated by differences between reflectance spectra. Details of a method for determining the state of the subject on the basis of differences between spectra will be described later.

The image forming unit 125 is a component that generates an image using information regarding the position at which the pulsed wave is radiated adjusted by moving the stage included in the sample unit 105 and the information obtained by the determination unit 124. The time waveform and the spectrum of the pulsed wave, the information regarding the subject, the image, and the like obtained by the above-described functions of the computer can be displayed on a display unit (not illustrated) connected to the computer as necessary.

The configuration of the apparatus 100 according to this embodiment is as described above. Now, the method for determining the state of the subject using the apparatus 100 will be described with reference to FIG. 2. FIG. 2 is a flowchart illustrating processes for determining the state of the subject using the apparatus 100 and a biological tissue as the subject and obtaining a two-dimensional image in which different colors are used in accordance with the state of the biological tissue.

The determination method can be divided into a first process, which determines a target region 22 of a subject 21 in which a state is determined, and a second process, which determines the state of the biological tissue over the entirety of the target region 22. Although the second process according to this embodiment includes a step of obtaining a two-dimensional image in which different colors are used in accordance with the state of the biological tissue on the basis of a result of the determination of the state of the biological tissue, this step may be omitted if the step is not necessary.

Here, the target region 22 is assumed to be a region including both a normal tissue portion and an abnormal tissue portion. The size of the target region 22 is a size that suits the biological tissue, which is the subject 21. For example, in the case of a hepatoma, the size of a tumor varies depending on the progression of the disease, but because the tumor is 20 mm or less in diameter in an initial stage, a target region of about 20×40 mm can include both the normal tissue portion and the abnormal tissue portion.

In the first process, which determines the target region 22, first, the control unit 121 selects, in the subject 21, a plurality of measurement point groups 30 to 34, each including two or more neighboring measurement points (S200). Next, a terahertz wave is radiated onto each of the selected measurement points in order for the obtaining unit 123 to obtain a reflectance spectrum at each measurement point (S201). Distances between the measurement point groups 30 to 34 are larger the beam diameters of the terahertz wave radiated onto the subject 21, and the arrangement of the plurality of measurement point groups 30 to 34 is randomly selected in a surface of the subject 21.

After the reflectance spectra are obtained for all the selected measurement points, the determination unit 124 obtains differences between the plurality of spectra obtained at the measurement points included in each of the measurement point groups 30 to 34 (S202). Next, the determination unit 124 determines the state of the subject 21 in each of the measurement point groups 30 to 34 on the basis of the differences between the plurality of spectra in each of the measurement point groups 30 to 34 obtained in the step of obtaining differences in step S202 (S203).

The “differences between the plurality of spectra” herein refer to differences between changes, which are caused in accordance with the frequency or the beam diameter, in the optical characteristics of the subject 21, the changes being indicated by the spectra obtained as a result of the measurement performed at the measurement points. The “changes in the optical characteristics of the subject 21 caused in accordance with the frequency or the beam diameter” refer to a way in which the optical characteristics of the subject 21 at a certain frequency or beam diameter change in accordance with changes in the frequency or the beam diameter. Therefore, the “differences between the plurality of spectra” refer to indices indicating how much the ways in which the spectra obtained at two or more measurement points change are different from each other, that is, indices indicating variation in the changes in the optical characteristics of the subject 21 depending on the frequency or the beam diameter. The “differences between the plurality of spectra” will also be referred to as “variation in the spectra” hereinafter, and these terms are synonymous.

The variation in the spectra tends to differ depending on where each of the measurement point groups 30 to 34 is located: a normal tissue portion 210, an abnormal tissue portion 211, or a boundary portion between the normal tissue portion 210 and the abnormal tissue portion 211. Therefore, which portion of the subject 21 each of the measurement point groups 30 to 34 is located in can be determined on the basis of the variation in the spectra. In this embodiment, differences between the shapes of the spectra are focused upon as the differences between the plurality of spectra (variation in the spectra). Details of a method for obtaining the variation in the spectra will be described later.

More specifically, variation in spectra tends to be largest in the abnormal tissue portion 211, moderate in the boundary portion, and smallest in the normal tissue portion 210. Therefore, the value of variation in spectra obtained for each of the measurement point groups 30 to 34 in the subject 21 is largest in the measurement point group 31, smallest in the measurement point groups 30 and 34, and moderate in the measurement point groups 32 and 33. That is, by comparing the values of variation in spectra obtained for the measurement point groups 30 to 34 and classifying the values into three categories, the state of the subject 21 can be determined.

Here, the value of variation in spectra obtained for the measurement point group 31 is stored as the value of the abnormal tissue portion 211, those obtained for the measurement point groups 30 and 34 are stored as the values of the normal tissue portion 210, and those obtained for the measurement point groups 32 and 33 are stored as the values of the boundary portion. In doing so, these values can be used in the second process.

If variation in spectra is substantially the same among all the selected measurement point groups 30 to 34, the entirety of the region can be the normal tissue portion 210 or the abnormal tissue portion 211. Therefore, the procedure from S201 to S203 is repeated by selecting a plurality of measurement point groups again in another region until variation in spectra differs between the measurement point groups and it is determined that both the normal tissue portion 210 and the abnormal tissue portion 211 are included in the region.

If a region including both the normal tissue portion 210 and the abnormal tissue portion 211 is found, the target region 22, in which the state is determined in the second process, is determined on the basis of the positions of the measurement point groups 30 to 34 and the results of the determination of the states of the measurement point groups 30 to 34 in the surface of the subject 21. If a multicolor image need not be obtained by determining the states, the operation may end here.

In the second process, in which the state of the target region 22 in the subject 21 is determined and a multicolor image is formed on the basis of a result of the determination, first, the target region 22 determined in the first process is subjected to two-dimensional line scan measurement (S205).

Distances between neighboring measurement points in the two-dimensional line scan measurement in this embodiment are 200 μm to 250 μm, which corresponds to ¼ to ⅕ of the beam diameters (up to about φ1 mm) of the terahertz wave. With this configuration, changes in the state of the biological tissue, which is an aggregate of cells of tens of micrometers to hundreds of micrometers in size, can be detected.

As in the first process, the determination unit 124 determines whether each measurement point group is classified into the normal tissue portion 210, the abnormal tissue portion 211, or the boundary portion of the biological tissue, which is the subject 21, on the basis of variation in spectra obtained at the plurality of measurement points. At this time, each measurement point group includes two or more neighboring measurement points on the same line. The determination unit 124 determines the state by comparing the variation in spectra obtained at the two or more neighboring measurement points on the same line with the values of the normal tissue portion 210, the abnormal tissue portion 211, and the boundary portion obtained in the first process.

For example, if the value of variation in the spectra obtained at three measurement points 24 is small and the same as or close to the values of variation in spectra obtained in the first process for the measurement point groups 30 and 34, the determination unit 124 determines that the corresponding measurement point group is the normal tissue portion 210 (S207). Similarly, because the value of variation in the spectra obtained at three measurement points 25 is large and falls into a range of values of variation obtained for the measurement point group 31, the determination unit 124 determines that the corresponding measurement point group is the abnormal tissue portion 211 (S208). In addition, because the value of variation in the spectra obtained at three measurement points 26 is close to the value of variation obtained for the measurement point group 32 or 33, the determination unit 124 determines that the corresponding measurement point group is the boundary portion (S209).

The determination unit 124 determines the states of all the measurement points in this manner. By displaying all the measurement points using colors corresponding to the states of the measurement points on the basis of the results of the determinations, the image forming unit 125 obtains an image 27 in which different colors are used in accordance with the state of the target region 22 of the biological tissue, which is the subject 21 (S210).

Although the values of variation in spectra corresponding to the states of the subject 21 obtained in the first process are used in steps S207, S208, and S209 in this embodiment, the state of the subject 21 can be determined without using these values. For example, as in the first process, the state of the subject 21 can be determined by forming a plurality of measurement point groups in the target region 22 subjected to the line scan measurement and comparing variation in spectra in each measurement point group.

If the two-dimensional line scan measurement in step S205 is performed without performing the first process, whether the target region 22 includes both the normal tissue portion 210 and the abnormal tissue portion 211 has not been determined. Therefore, the first process is desirably performed if it is desired that both the normal tissue portion 210 and the abnormal tissue portion 211 be included in the target region 22.

Thus, according to the apparatus 100 according to this embodiment, the state of the subject 21 can be determined, without referring to a database, on the basis of variation in spectra obtained by radiating terahertz waves onto three or more measurement points.

Now, as an outline of the method for determining the state of the subject 21, the reason why variation in spectra differs depending on the state of a biological tissue and a method for obtaining variation in spectra will be described.

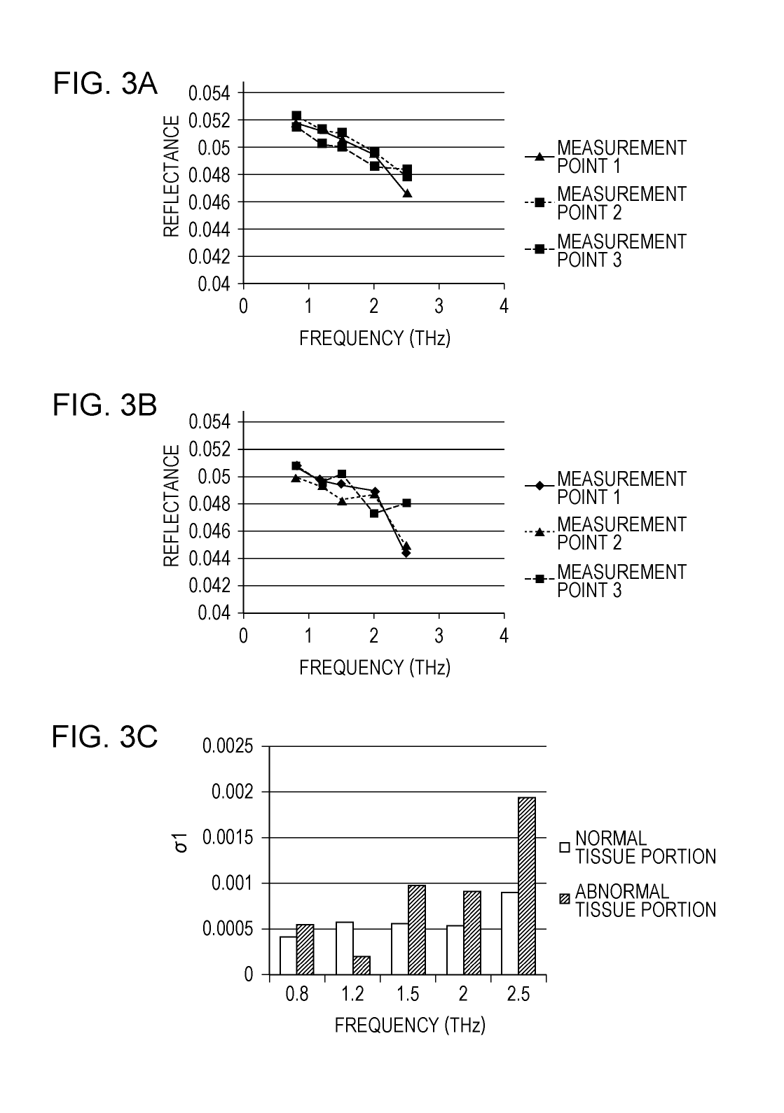

FIG. 3A illustrates reflectance spectra obtained by measuring a normal tissue portion of a paraffin-embedded human liver fixed block at three measurement points using the apparatus 100. FIG. 3B illustrates reflectance spectra obtained by measuring an abnormal tissue portion of the paraffin-embedded human liver fixed block at three measurement points using the apparatus 100. A horizontal axis represents frequency, and a vertical axis represents reflectance for the reflectance spectra. Results of the measurement are plotted at five frequencies of 0.8, 1.2, 1.5, 2.0, and 2.5 THz. FIG. 3C illustrates, as standard deviations (al), variation (differences between spectra) in reflectance at each frequency using the reflectance spectra illustrated in FIGS. 3A and 3B.

In FIG. 3C, the standard deviations σ1 are larger in the abnormal tissue portion than in the normal tissue portion except at the frequency of 1.2 THz. In addition, whereas changes in the standard deviation σ1 depending on the frequency are small in the normal tissue portion, changes in the standard deviation σ1 depending on the frequency are large. That is, how the reflectance changes in accordance with the frequency is inconsistent.

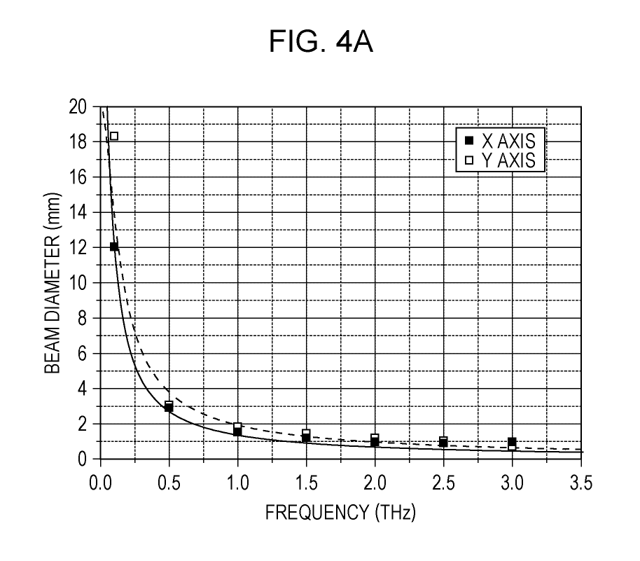

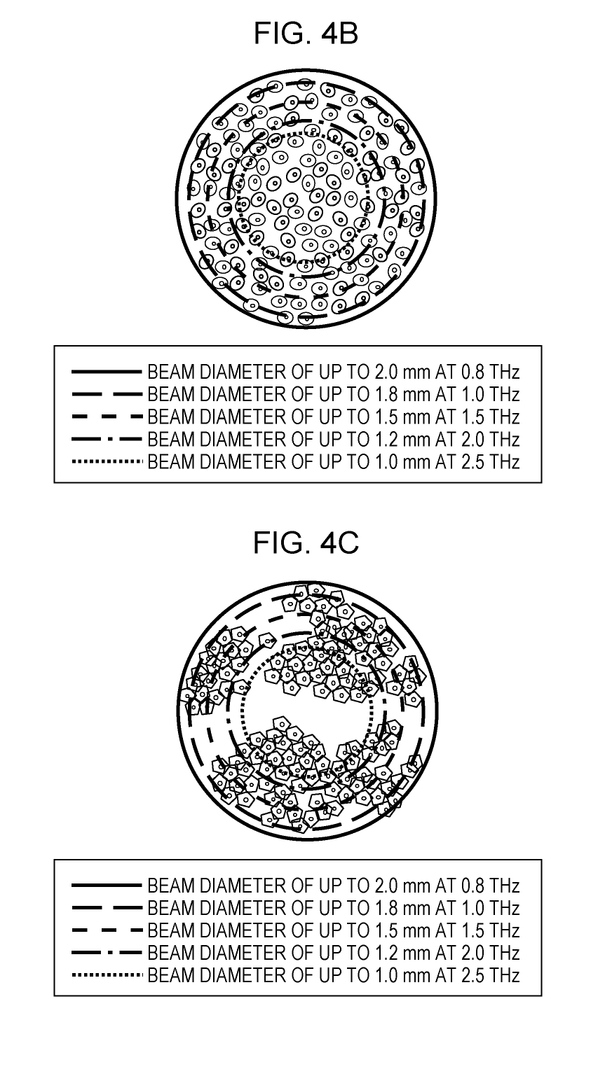

One of major factors in the large variation in the spectra in the abnormal tissue portion is a relationship between the beam diameters of the terahertz wave and the sizes of cells in the biological tissue. FIG. 4A illustrates the frequency dependence of the beam diameters of the terahertz wave in the apparatus 100. In FIG. 4A, the terahertz wave propagates along an X axis, and a direction perpendicular to the propagation direction is a Y axis.

Since the terahertz wave in this embodiment is a pulsed wave, the terahertz wave includes a plurality of different frequency components. In a terahertz region, in which the wavelength is long (300 μm at 1 THz), the beam diameters of the terahertz wave are on the order of millimeters and change on the order of sub-millimeters. On the other hand, the size of each cell included in the biological tissue is tens of micrometers to hundreds of micrometers. That is, a terahertz wave whose beam diameters are on the order of millimeters is radiated onto an aggregate of cells, not each cell, and the size of an observed region, that is, the beam diameters, differs depending on the frequency.

In the apparatus 100 adopting the THz-TDS, light having a plurality of frequencies and different beam diameters is focused such that the centers of the beam diameters match. Therefore, even if the frequencies or the beam diameters are different, the centers of the beam diameters substantially match, and therefore differences in spectra of a biological tissue caused by various frequencies and beam diameters can be compared with one another.

In the abnormal tissue portion 211 of the biological tissue, nucleic acids increase because cells are in a proliferation phase, and cell nuclei become larger than those in the normal tissue portion 210. In addition, in the abnormal tissue portion 211, intussuscepted cells and disorder in the arrangement of cells are observed more often than in the normal tissue portion 210, and the cells tend to cluster together.

FIG. 4B is a diagram schematically illustrating a terahertz beam radiated onto the normal tissue portion 210, in which cells are homogeneously distributed without intussuscepted cells or disorder in the arrangement of the cells. FIG. 4C is a diagram schematically illustrating a terahertz beam radiated onto the abnormal tissue portion 211, in which cells are inhomogeneously distributed and cluster together in some portions. Stroma including fiber components, such as collagen fiber, and glycoprotein fills portions where there are no cells.

In the abnormal tissue portion 211, in which the cells are inhomogeneously distributed, the percentage contents of the cells and the stroma greatly change depending on the beam diameter, compared to those in the normal tissue portion 210, in which the cells are homogeneously distributed. These differences in the percentage contents of the cells and the stroma depending on the beam diameter are reflected by the optical characteristics in the terahertz region such as reflectance or transmittance at each frequency, and, as a result, the shapes of spectra differ between the normal tissue portion 210 and the abnormal tissue portion 211.

Furthermore, whereas the distribution of cells does not significantly vary between measurement points in the normal tissue portion 210 of the same subject, the distribution of cells vary between measurement points in the abnormal tissue portion 211, in which the cells are inhomogeneously distributed, even if the same subject is used.

Therefore, the shapes of spectra in the terahertz region obtained from the abnormal tissue portion 211 of the biological tissue are different from those obtained from the normal tissue portion 210, and the shapes of spectra are also different between measurement points. That is, since the shapes of spectra in the terahertz region obtained from the biological tissue at measurement points are different between the normal tissue portion 210 and the abnormal tissue portion 211, the state of the biological tissue can be determined on the basis of variation in the spectra.

In addition to changes in the percentage contents depending on the beam diameter, scattering depending on the frequency can be a factor in variation in spectra. As described above, in the abnormal tissue portion 211 of the biological tissue, cells tend to be inhomogeneously distributed and cluster together in some portions. Since the size of each cell is tens of micrometers to hundreds of micrometers, the size of each cell cluster is 100 μm to hundreds of micrometers. The wavelength of a terahertz wave is 300 μm at 1 THz, and if there is a particle as large as the wavelength, Mie scattering occurs. Therefore, if the abnormal tissue portion 211 includes a cell cluster of hundreds of micrometers, which corresponds to the wavelength of a terahertz wave, the terahertz wave scatters and affects the shape of a resultant spectrum.

In the case of reflection or transmission measurement, a terahertz wave scattered by the subject 21 does not reach the detection unit 107 and accordingly is not detected. Therefore, the reflectance or the transmittance at a frequency affected by the scattering becomes abnormally low in a reflectance spectrum or a transmittance spectrum. More specifically, if the size of a cell cluster is about 100 μm, the reflectance or the transmittance becomes low on a high frequency side including a terahertz wave of about 3 THz, and if the size of a cell cluster is about 300 μm, the reflectance or the transmittance becomes low on a high frequency side including a terahertz wave of about 1 THz.

A frequency band in which the value of the optical characteristics such as the reflectance or the transmittance decreases shifts depending on the size of a cell cluster, which acts as a scatterer, that is, the frequency band shifts to a low frequency side as the scatterer becomes larger. Because the abnormal tissue portion 211 includes cell clusters of various sizes, variation in spectra can be likely caused by the scattering in the abnormal tissue portion 211, compared to the normal tissue portion 210.

The apparatus 100 according to this embodiment is configured such that light having a plurality of frequencies and different beam diameters is focused to the same point. That is, the beam diameters and the frequencies simultaneously change. In the abnormal tissue portion 211, in which cells tend to be inhomogeneously distributed and cluster together in some portions, the percentage contents greatly change depending on the beam diameter compared to those in the normal tissue portion 210, and accordingly variation in spectra between the measurement points becomes large. If the abnormal tissue portion 211 includes a cell cluster as large as the wavelength of a terahertz wave radiated onto the subject 21, scattering is likely to occur on a low frequency side with a relatively large cell cluster and on a high frequency side with a relatively small cell cluster. Therefore, variation in spectra between the measurement points becomes larger than in the normal tissue portion 210.

That is, in this embodiment adopting the THz-TDS apparatus 100, how cells cluster together in the abnormal tissue portion 211 can be identified on the basis of both the amount of change in the percentage contents of components and the amount of scattering caused by cell clusters as large as the wavelength of a terahertz wave.

In the method for determining the state of a biological tissue on the basis of variation in spectra using the apparatus 100 according to this embodiment, it is important that the inhomogeneous distribution of cells in the abnormal tissue portion 211 is as large as the wavelength of a terahertz wave.

The apparatus 100 obtains a spectrum in any frequency region in a frequency region of 30 GHz to 30 THz, and an observed region, which corresponds to the beam diameter changes on the order of sub-millimeters depending on the frequency. Therefore, if there is one reflectance spectrum at each measurement point, the state of a biological tissue including inhomogeneous distribution on the order of sub-millimeters can be determined.

If light having a short wavelength compared to the sizes of cells included in a biological tissue is used, it is difficult to radiate light having a plurality of beam diameters that are different on the order of sub-millimeters in one measurement and determine the state of the biological tissue using a single reflectance spectrum.

A difference in the percentage contents of the components of the biological tissue, which is the subject 21, between the normal tissue portion 210 and the abnormal tissue portion 211 will be described in detail. The reflectance spectra obtained from a paraffin-embedded human liver fixed block have been described with reference to FIGS. 3A and 3B. In the case of a paraffin-embedded fixed block, substantially all the stroma of the subject is replaced by paraffin, and accordingly the components of the biological tissue are the following three components: cell nuclei, cytoplasm, and paraffin.

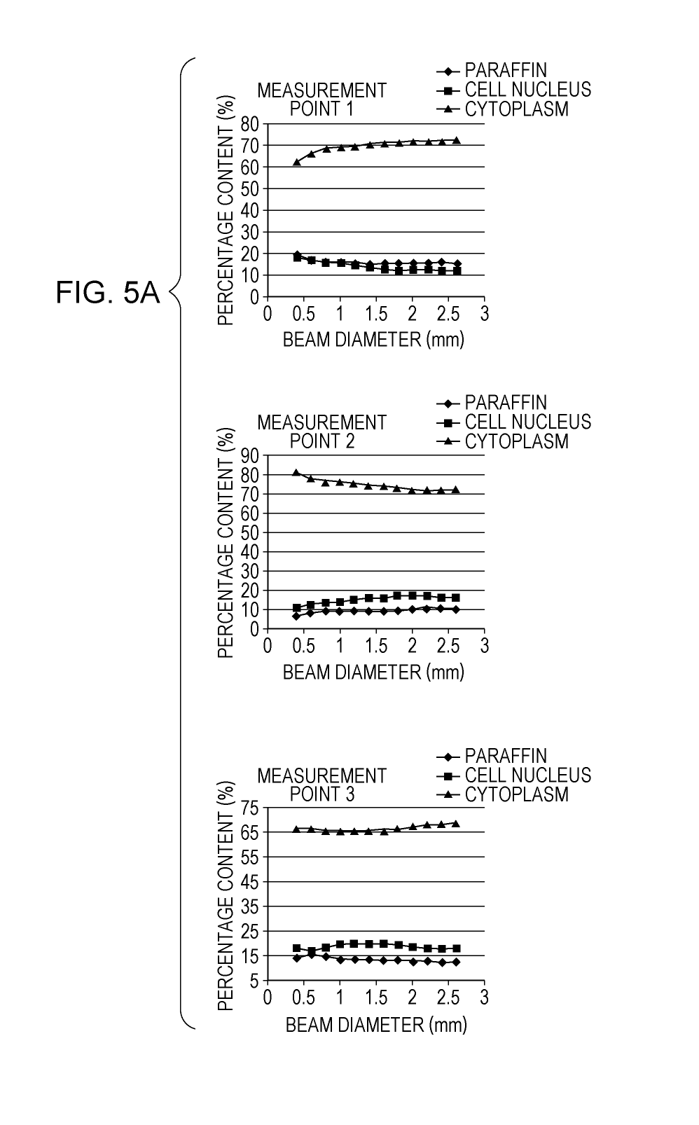

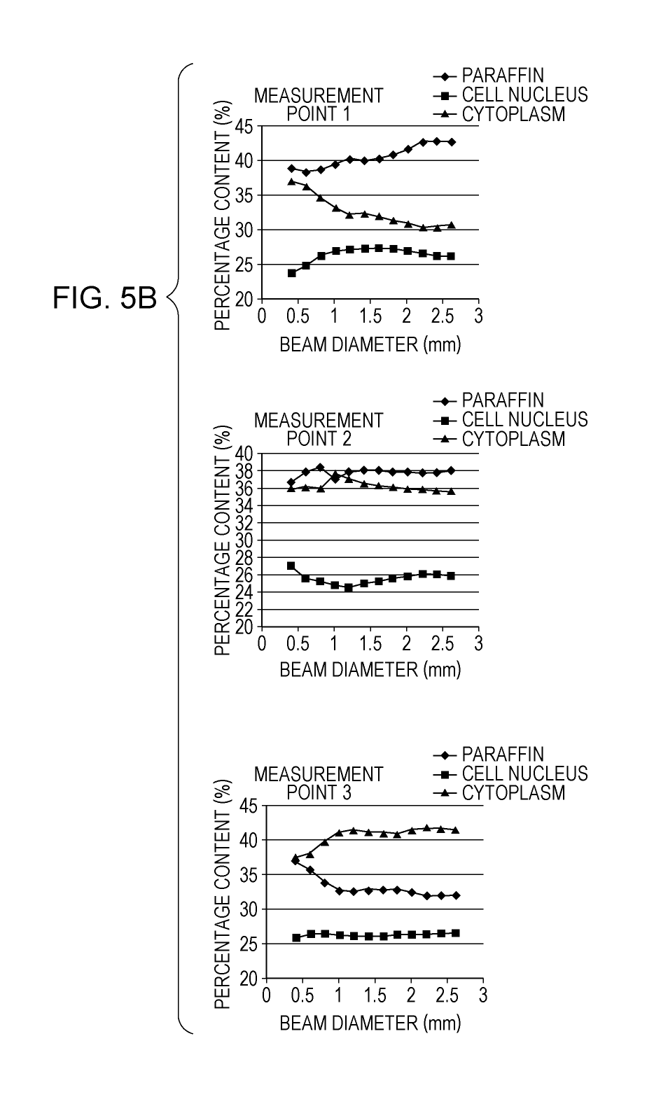

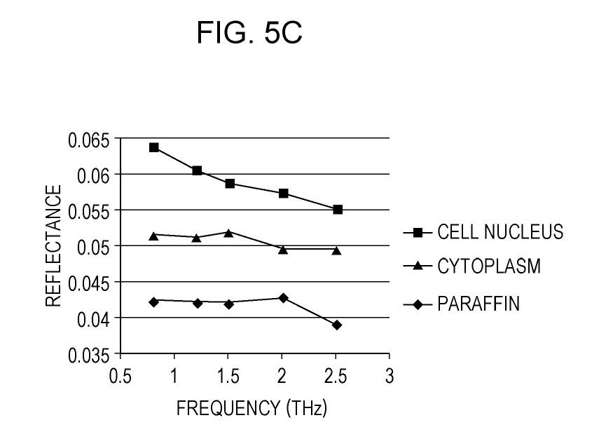

FIG. 5A includes graphs illustrating relationships between the percentage contents of the three components and the beam diameter at three measurement points in the normal tissue portion 210 of the paraffin-embedded human liver fixed block used for measuring the reflectance spectra. FIG. 5B includes graphs illustrating relationships between the percentage contents of the three components and the beam diameter at three measurement points in the abnormal tissue portion 211 of the paraffin-embedded human liver fixed block used for measuring the reflectance spectra. These results are calculated from analyses of HE-stained images of the paraffin-embedded human liver fixed block.

Each HE-stained image is obtained by capturing an image of a 3 μm thick stained slice of the paraffin-embedded human liver block used for measuring the reflectance spectra. In each HE stained image, portions stained by hematoxylin mainly correspond to the cell nuclei, portions stained by eosin mainly correspond to the cytoplasm, and portions that are not stained correspond to the paraffin of the paraffin-embedded fixed block. Therefore, by analyzing the HE stained image using various beam diameters at the measurement points, the percentage contents of the three components can be calculated.

It can be seen from FIG. 5A that the percentage contents of the cytoplasm are high at the three measurement points in the normal tissue portion 210 and the percentage contents of the three components do not significantly vary between the measurement points. In addition, the percentage contents do not significantly vary depending on the beam diameter. This indicates, as described above, that the distribution of cells in the normal tissue portion 210, in which cells are homogeneously distributed, of the same subject 21 is not significantly different between the measurement points.

On the other hand, it can be seen from FIG. 5B that the percentage contents of the three components at the three measurement points in the abnormal tissue portion 211 are different from those at the three measurement points in the normal tissue portion 210 and how the percentage contents change depending on the beam diameter is different at each measurement point. This indicates, as described above, that the distribution of cells in the abnormal tissue portion 211, in which cells are inhomogeneously distributed, is different between the measurement points even if the same subject 21 is used.

FIG. 5C illustrates the reflectance spectrum of a cell nucleus region and the reflectance spectrum of a cytoplasm region obtained by comparing results of the analyses of the HE-stained images and the reflectance spectra and the separately obtained reflectance spectrum of the paraffin. It can be seen from FIG. 5C that the values of the reflectance spectra of the three components are different from one another. Therefore, if the percentage components of the three components are different, the values of the reflectance spectra obtained as a result of the measurement are also different.

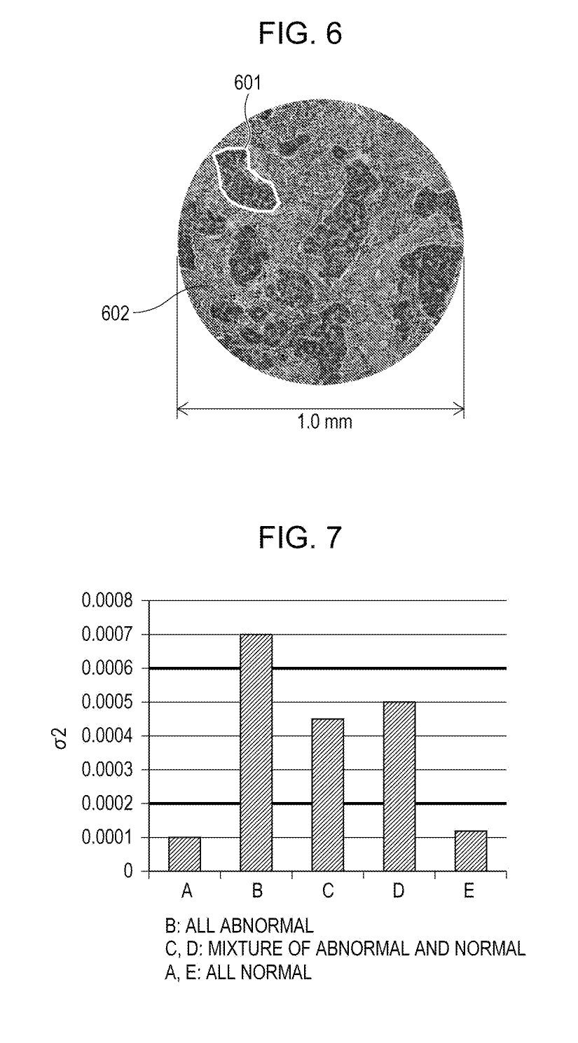

FIG. 6 illustrates an HE-stained image obtained at one of the three measurement points at which the reflectance spectra of the abnormal tissue portion 211 illustrated in FIG. 3B have been obtained. FIG. 6 illustrates a region of 1.0 mm in diameter, which corresponds to the beam diameter at 2.5 THz. The abnormal tissue portion 211 includes a plurality of regions 601 in which cancerous liver cells cluster together and a region 602 filled with stroma including inflammatory cells. In the abnormal tissue portion 211, dark-colored elliptical regions including the regions 601 are inhomogeneously distributed. Thus, in the abnormal tissue portion 211, in which cells inhomogeneously cluster together on the order of sub-millimeters, which corresponds to the wavelength of a terahertz wave, the values of physical properties such as the reflectance and the transmittance decrease because of the scattering, which affects lower frequencies as the sizes of cell clusters become larger. Since the sizes of the cell clusters vary, the shapes of the spectra can also vary.

Thus, the state of a biological tissue can be determined on the basis of variation in spectra in the terahertz region obtained from the biological tissue.

Next, an example of a method for obtaining variation in spectra will be described. As described above, FIG. 3C is a histogram representing the variation in the reflectance spectra illustrated in FIGS. 3A and 3B as the standard deviations (σ1) of the three measurement points at each frequency. Similarly, the standard deviations σ1 at each frequency can be obtained for the plurality of measurement point groups 30 to 34 selected in step S200.

The standard deviations σ1 of the measurement point groups 30 and 34 included in the normal tissue portion 210 are close to that of the three measurement points in the normal tissue portion 210 illustrated in FIG. 3C. The standard deviation σ1 of the measurement point group 31 is close to that of the three measurement points in the abnormal tissue portion 211 illustrated in FIG. 3C. The standard deviations σ1 of the measurement point groups 32 and 33, which are included in the boundary portion, are a mixture of the standard deviation σ1 of the three measurement points in the normal tissue portion 210 and the standard deviation σ1 of the three measurement points in the abnormal tissue portion 211.

Here, a second standard deviation σ2 may be obtained for the standard deviations σ1 (hereinafter referred to as a first standard deviation σ1) at the plurality of frequencies obtained for each measurement point group and may be used as differences between the plurality of spectra. As described above, since the shape of a spectrum obtained in the abnormal tissue portion 211 is different between the measurement points, variation in the value of the first standard deviation σ1, that is, the second standard deviation σ2, is large if a measurement point group includes a measurement point in the abnormal tissue portion 211. Therefore, the second standard deviation σ2 may be used as an index for estimating variation in spectra for distinguishing the normal tissue portion 210 and the abnormal tissue portion 211.

FIG. 7 illustrates an example of the second standard deviations σ2 obtained from the measurement point groups 30 to 34 illustrated in FIG. 2. The largest second standard deviation σ2 is obtained from the measurement point group 31, which is included in the abnormal tissue portion 211, and the smallest second standard deviation σ2 is obtained from the measurement point groups 30 and 34, which are included in the normal tissue portion 210. In the measurement point groups 32 and 33, which are included in the boundary portion between the normal tissue portion 210 and the abnormal tissue portion 211, the standard deviations σ2 are moderate values.

In the example illustrated in FIG. 7, if the second standard deviation σ2 is 0.0006 or larger, the corresponding measurement point group is included in the abnormal tissue portion 211. If the second standard deviation σ2 is larger than 0.0002 but smaller than 0.0006, the corresponding measurement point group is included in the boundary portion, and if the second standard deviation σ2 is 0.0002 or smaller, the corresponding measurement point group is included in the normal tissue portion 210. Thus, the second standard deviation σ2 may be used as an index for determining the state of the subject 21 in the first process and the second process illustrated in FIG. 2 and using different colors in the second process.

As illustrated in FIG. 6, in the abnormal tissue portion 211, in which cells inhomogeneously cluster together on the order of sub-millimeters, the percentage contents of the components can change within a range of several percent and the effect of the scattering can be reflected if the beam diameter changes. Therefore, the state of the subject 21 can be determined using an index indicating variation in spectra, such as the second standard deviation σ2.

As described above, in the case of a biological tissue, which is an aggregate of cells of tens of micrometers to hundreds of micrometers in size, inhomogeneousness in the cells in the abnormal tissue portion 211 is on the order of sub-millimeters. This holds not only for a hepatoma in the liver but also for an adenocarcinoma in the large intestine or the stomach. In the case of an adenocarcinoma in the large intestine or the stomach, disorder on the order of sub-millimeters occurs in the shapes of mucosal epithelial cells in the abnormal tissue portion 211 and the shapes of lamina propria mucosae, which are stroma in mucosae, compared to a mucosal layer in the normal tissue portion 210. As in a hepatoma, the disorder is inhomogeneous depending on the position. Therefore, the subject 21 whose state can be determined on the basis of variation in spectra obtained using terahertz waves whose beam diameters are on the order of millimeters is not limited to the liver but may be any biological tissue in which inhomogeneousness occurs in an abnormal tissue portion on the order of sub-millimeters.

In order to determine the state of a biological tissue, frequencies in the terahertz region used for obtaining variation are desirably frequencies at which the beam diameter is 0.5 mm to 3.0 mm, with which the inhomogeneousness of the components on the order of sub-millimeters can be observed. In order to detect variation caused by scattering, the frequency band desirably includes a band of 3.0 THz or shorter, which corresponds to a wavelength of about 100 μm or longer expected as the sizes of cell clusters.

At least two frequencies are needed to obtain variation in spectra, and the number of frequencies whose beam diameters are different needs to be increased in order to increase the accuracy of the determination. The bands and the frequency resolution of spectra obtained by the apparatus 100 depend on the generation device used, the detection device used, the sweep distance of a delay optical system at a time when a time waveform is obtained, and the like, but spectra of up to several terahertz can be obtained with a resolution of tens of gigahertz. Therefore, it is easy to add frequencies.

The subject 21 used in the apparatus 100 and the determination method according to this embodiment is not limited to a biological tissue, but may be a substance other than a biological tissue having inhomogeneousness on the order of sub-millimeters. For example, this embodiment may be applied to a determination of the distribution of pores in a metal, a ceramic, a resin, or the like including the pores on the order of sub-millimeters.

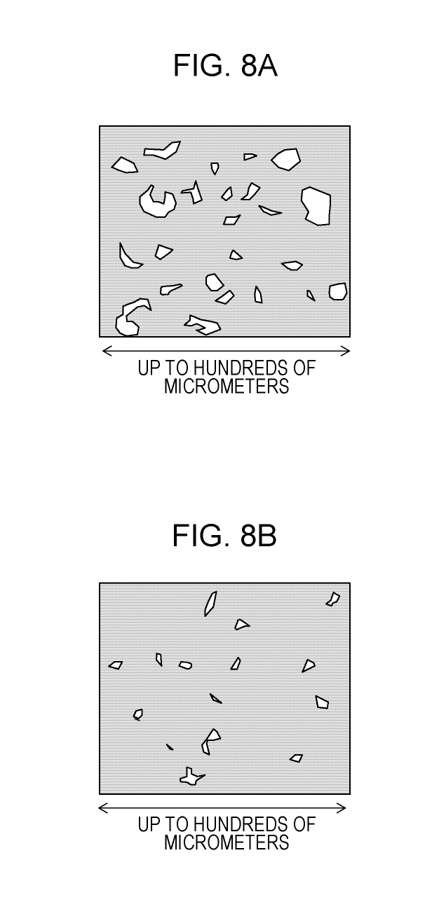

There are various ceramic porous bodies including pores of several angstroms to several millimeters for various uses. FIG. 8A is a cross-sectional view of a ceramic porous body including pores on the order of sub-millimeters and the area occupied by the pores is large, and FIG. 8B is a cross-sectional view of a ceramic porous body including pores smaller than sub-millimeters and the area occupied by the pores is small.

Using the same procedure as in the above-described method for determining the state of a biological tissue, a plurality of measurement point groups can be selected in each of the ceramic porous bodies illustrated in FIGS. 8A and 8B and the second standard deviations σ2 can be calculated as variation in reflectance spectra.

If there are inhomogeneously distributed pores on the order of sub-millimeters and the area occupied by the pores is large, the pores are likely to be included in the beam diameters of a terahertz wave, and the area occupied by the pores differs at each measurement point. In the case of a ceramic porous body, spectra obtained using terahertz waves are different between a ceramic region and a pore region. Therefore, if the area occupied by the pores is large, variation in the spectra, which are obtained using terahertz waves, at the measurement points becomes different. On the other hand, if there are pores smaller than sub-millimeters and the area occupied by the pores is small, it is unlikely that the beam diameters of a terahertz wave include pores, and accordingly the area occupied by the pores does not significantly vary between the measurement points. Therefore, if the area occupied by the pores is small, there is no significant difference in the variation of the spectra, which are obtained using terahertz waves, at the measurement points.

Thus, by radiating terahertz waves onto a plurality of measurement point groups in a ceramic porous body, obtaining the second standard deviations σ2 for obtained reflectance spectra, and comparing the second standard deviations σ2, a difference in the area occupied by the pores can be detected. In other words, using the same procedure as that used in the determination method illustrated in FIG. 2, the distribution of pores in a metal, a ceramic, a resin, or the like including pores on the order of sub-millimeters can be determined on the basis of a difference in the area occupied by the pores.

According to the apparatus 100 in this embodiment and the determination method using the apparatus 100, the state of the subject 21 can be determined without referring to a database.

### Second Embodiment

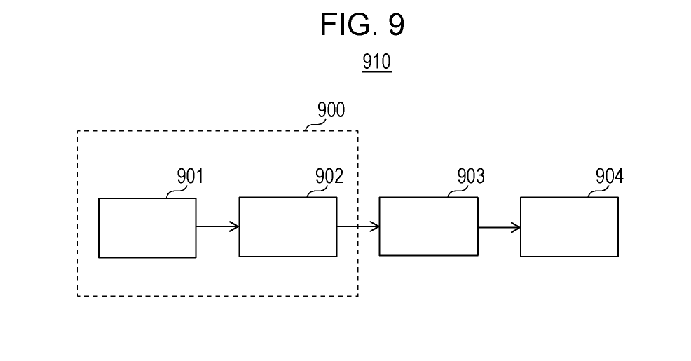

An information obtaining apparatus 910 (hereinafter referred to as an “apparatus 910”) according to a second embodiment will be described with reference to FIG. 9. The apparatus 100 according to the first embodiment obtains the time waveform of a terahertz wave using terahertz wave pulses. In this embodiment, however, the beam diameters of a continuous terahertz wave having a plurality of different frequencies are modulated into beam diameters corresponding to the frequencies, and measurement is performed by radiating the obtained terahertz wave having the plurality of different beam diameters onto a subject. Description of the same components as those according to the first embodiment is omitted. The apparatus 910, too, includes the storage unit 126 storing the programs corresponding to the steps illustrated in the flowchart of FIG. 2, and each process is performed by the CPU, which reads and executes each of the programs.

FIG. 9 is a diagram illustrating the configurations of a radiation unit 900 and a detection unit of the apparatus 910. The radiation unit 900 according to this embodiment includes a continuous wave light source 901 including a light source that generates a continuous wave having a plurality of different frequencies and a beam diameter modulation section 902. The terahertz continuous wave generated by the continuous wave light source 901 is modulated by the beam diameter modulation section 902 in such a way as to obtain various beam diameters and radiated onto a subject in a sample unit 903. Thereafter, the terahertz wave that has penetrated the subject or that has been reflected from the subject is detected by a detector 904 as the detection unit.

As the continuous wave light source 901 that generates the terahertz continuous wave, a resonant-tunneling diode oscillator, a quantum cascade laser, or the like may be used. The beam diameter modulation section 902 prepares a unit for changing the beam diameters for each of the different frequencies. More specifically, the beam diameters can be modulated by, for example, adjusting focal distances between mirrors and lenses used for focusing the continuous wave having the different frequencies. If a plurality of light sources that generate continuous waves having different frequencies are prepared, a unit for aligning the centers of beams of the plurality of frequencies is necessary. As the detector 904, a complementary metal-oxide-semiconductor (CMOS) detector, a Schottky detector, or the like may be used.

In this embodiment, by preparing two or more light sources that generate continuous waves having different frequencies, the second standard deviation σ2 of each measurement point group can be obtained. That is, because the optical characteristics with each beam diameter can be obtained by radiating the terahertz waves having the plurality of different beam diameters onto the subject, the state of a biological tissue can be determined on the basis of variation in spectra using the same sequence as that according to the first embodiment.

In this embodiment, measurement is performed by modulating two or more terahertz continuous waves having different frequencies in such a way as to obtain beam diameters according to the frequencies and radiating the terahertz continuous waves onto the subject. Alternatively, measurement may be performed by modulating the beam diameters of terahertz continuous waves having the same frequency may be modulated and radiating the terahertz continuous waves onto the subject. In this case, too, variation in how the optical characteristics of the subject change depending on the beam diameter can be identified and the state of the subject can be determined by obtaining data regarding reflectance spectra drawn along a horizontal axis representing beam diameter. The reflectance data obtained here for a normal tissue portion and an abnormal tissue portion corresponds to data obtained by removing the effect of scattering depending on the frequency from the spectra illustrated in FIGS. 3A and 3B and replacing the horizontal axis with one representing beam diameter.

Alternatively, measurement may be performed by radiating terahertz continuous waves having different frequencies and the same beam diameter onto the subject. In this case, variation in how the optical characteristics of the subject change depending on a difference in the effect of the scattering depending on the frequency can be identified. The reflectance data obtained here for the normal tissue portion and the abnormal tissue portion is drawn along a horizontal axis representing frequency and similar to the spectra illustrated in FIGS. 3A and 3B, but since the beam diameter is the same among the different frequencies, the reflectance data does not reflect changes in the components.

According to the apparatus 910 according to this embodiment and the determination method using the apparatus 910, the state of the subject can be determined without referring to a database.

### First Example

As a first example, the apparatus 100 according to the first embodiment will be described more specifically. In this example, a biological tissue is used as the subject, and a paraffin-embedded human liver fixed block is used as the biological tissue. Measurement is performed using pulsed waves including terahertz waves having five frequencies (0.8, 1.2, 1.5, 2.0, and 2.5 THz), and the second standard deviation σ2 is obtained as variation in spectra from results of the detection using the terahertz waves.

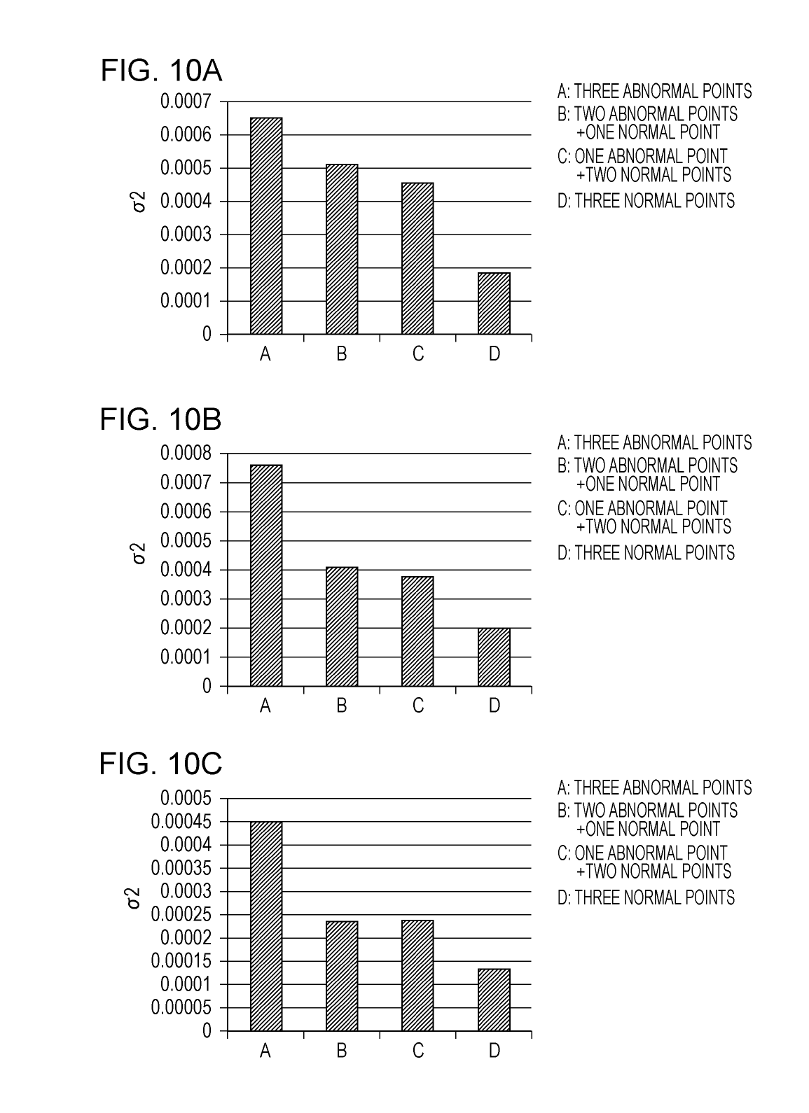

FIG. 3C illustrates, as the standard deviations σ1, variation in reflectance at each frequency using the reflectance spectra illustrated in FIGS. 3A and 3B. FIG. 10A illustrates a second standard deviation σ2 obtained, as an index indicating variation in spectra, from the first standard deviations σ1 at the three measurement points at each frequency in each of the normal tissue portion and the abnormal tissue portion. The second standard deviation σ2 of the three measurement points in the normal tissue portion is indicated by D, and the second standard deviation σ2 of the three measurement points in the abnormal tissue portion is indicated by A.

In FIG. 10A, the second standard deviations σ2 obtained by measuring two measurement point groups, namely a measurement point group including two measurement points in the abnormal tissue portion and one measurement point in the normal tissue portion and a measurement point group including one measurement point in the abnormal tissue portion and two measurement points in the normal tissue portion, are indicated as B and C, respectively.

As described above, since the shape of a spectrum is different between the measurement points in the abnormal tissue portion, if a measurement point in the abnormal tissue portion is included in a combination of measurement points with which the first standard deviation σ1 is calculated, variation in the first standard deviation σ1, that is, the second standard deviation σ2, increases. Therefore, the second standard deviation σ2 may be used as an index for estimating variation in spectra for distinguishing the normal tissue portion and the abnormal tissue portion.

As illustrated in FIG. 10A, the second standard deviation σ2 is indeed largest when all the three measurement points are included in the normal tissue portion (A) and smallest when all the three measurement points are included in the normal tissue portion (D). In addition, when the three measurement points are included in both the abnormal tissue portion and the normal tissue portion (B, C), the second standard deviation σ2 is a moderate value. This holds for another subject. FIGS. 10B and 10C illustrate second standard deviations σ2 obtained, using the same method as above, by measuring two subjects extracted from individuals different from one from which the subject used for obtaining the second standard deviations σ2 illustrated in FIG. 10A has been extracted. Although there are some differences in the second standard deviation σ2 depending on the subject, how the second standard deviation σ2 varies remains the same.

The state of the subject can be determined by obtaining the second standard deviation σ2 for each measurement point group on the basis of this variation in the second standard deviation σ2. More specifically, if the subject is a paraffin-embedded human liver fixed block, a measurement point group of a second standard deviation σ2 of 0.0002 or smaller can be determined as the normal tissue portion, and a measurement point group of a second standard deviation σ2 more than three times larger than that obtained from the normal tissue portion can be determined as the abnormal tissue portion. In addition, a measurement point group of a second standard deviation σ2 between the above two ranges can be determined as the boundary portion.

A relationship between the first standard deviation σ1 and the amount of change in the percentage contents of the components of biological tissues in the human liver fixed blocks of the three subjects will be described. The first standard deviation σ1 reflects differences in the percentage contents of the components of the biological tissues at each measurement point at each frequency.

In the combination of the three measurement points in the normal tissue portion illustrated in FIG. 3C, the first standard deviation σ1 remains at about 0.0005 regardless of the frequency, except at a frequency of 2.5 THz. According to the reflectance spectra of the three components illustrated in FIG. 5C, the amount of change in the percentage contents at a time when the first standard deviation σ1 is 0.0005 is ±0.9% in cell nuclei, ±1.0% in cytoplasm, and ±1.2% in paraffin.

In the measurement point group of the combination of the three measurement points in the abnormal tissue portion, the first standard deviation σ1 is 0.002, which is the largest value, at a frequency of 2.5 THz, at which the beam diameter is about 1.0 mm, and 0.0002, which is the smallest value, at a frequency of 1.2 THz, at which the beam diameter is about 1.5 mm. The amount of change in the percentage contents in the former case is ±4.0% in cell nuclei, ±4.5% in cytoplasm, and ±5.5% in paraffin. The amount of change in the percentage contents in the latter case is ±0.4% in cell nuclei, ±0.4% in cytoplasm, and ±0.5% in paraffin.

That is, the second standard deviations σ2 illustrated in FIG. 10A serve as indices for distinguishing the normal tissue portion, in which the amount of change in the percentage contents of cell nuclei, cytoplasm, and paraffin at each measurement point is about ±1.0%, and the abnormal tissue portion, in which the amount of change in the percentage contents of cell nuclei, cytoplasm, and paraffin at each measurement point is ±0.4% to ±5.5%. These ranges vary depending on the subject, but if the ranges are those described in this example, the state of a subject in which the percentage contents of the components at each measurement point change within a range of several percent can be determined.

As described above, according to the apparatus 100 according to this example and the determination method using the apparatus 100, the state of the subject can be determined without referring to a database.

### Second Example

As a second example, another example of the apparatus 100 according to the first embodiment will be described more specifically. Although a human liver fixed block embedded in paraffin 1203 is used as a biological tissue as the subject in the first example, a paraffin-embedded human large intestine fixed block is used as a biological tissue as the subject in this example. Measurement is performed using pulsed waves including terahertz waves having five frequencies (0.8, 1.0. 1.2, 1.6, and 2.0 THz), and the second standard deviation σ2 is obtained as variation in spectra from results of the detection using the terahertz waves.

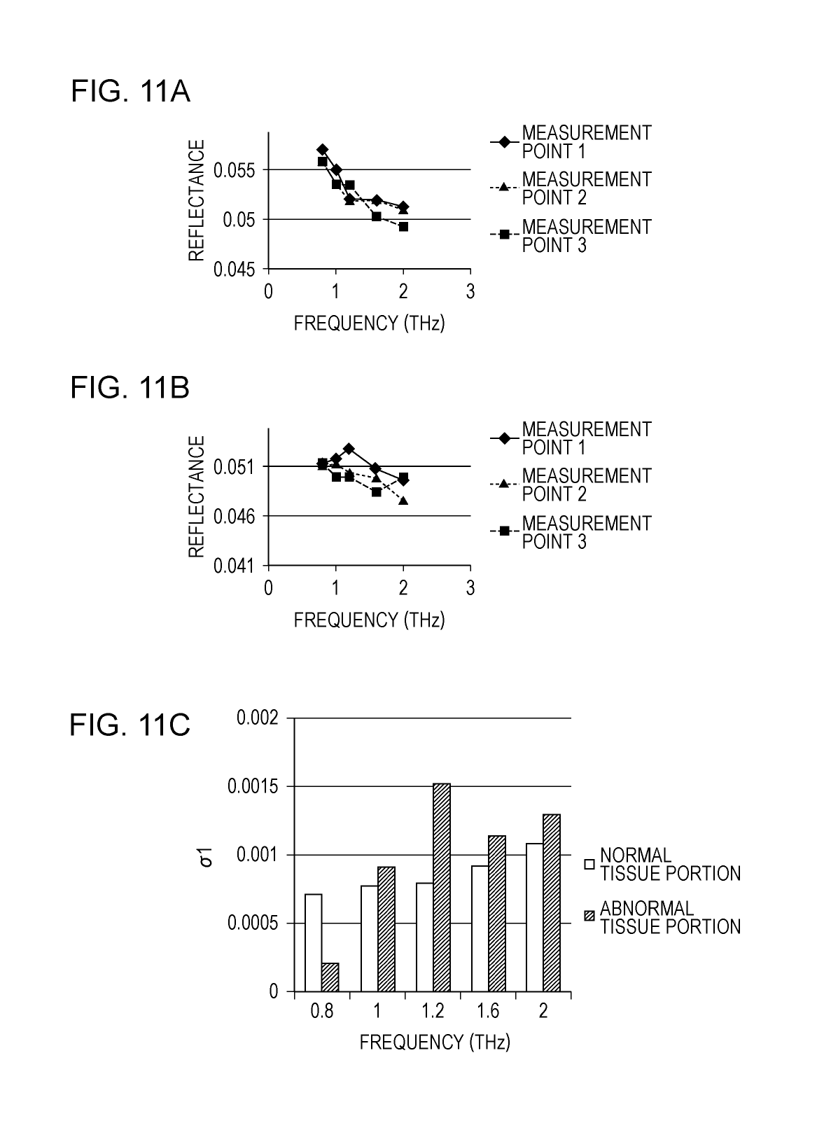

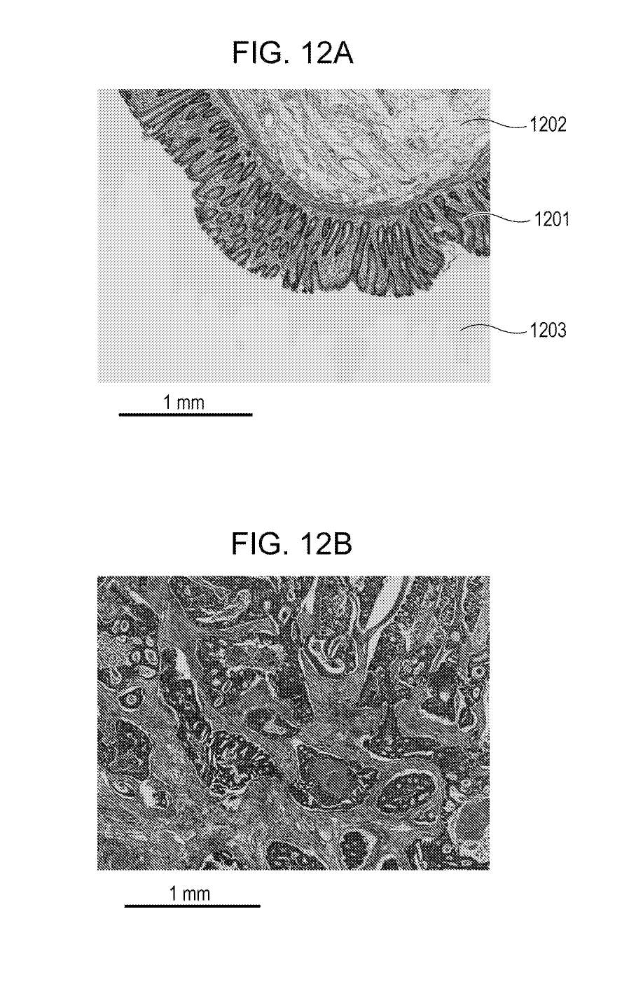

The abnormal tissue portion in this example is a large intestine adenocarcinoma formed in a mucosal layer of the large intestine. FIG. 11A illustrates reflectance spectra, which are obtained at three measurement points in the normal tissue portion of the paraffin-embedded human large intestine fixed block, drawn along a horizontal axis representing frequency. FIG. 11B illustrates reflectance spectra, which are obtained at three measurement points in the abnormal tissue portion of the paraffin-embedded human large intestine fixed block, drawn along a horizontal axis representing frequency. FIG. 12A illustrates an HE-stained image of a portion around a normal mucosal layer 1201 of the large intestine, and FIG. 12B illustrates an HE-stained image of a large intestine adenocarcinoma region. Whereas the thickness of the normal mucosal layer 1201 of the large intestine is about 1 mm, the thickness of the abnormal tissue portion is several to tens of millimeters because of hyperplasia of cancerous cells.

As described above, the beam diameters of a terahertz wave are on the order of sub-millimeters. Therefore, in a frequency band in which the beam diameter is larger than the thickness of the normal mucosal layer 1201, a spectrum obtained at a measurement point in the normal mucosal layer 1201 is affected by the region of a submucosal layer 1202 and the region of the paraffin 1203 adjacent to the normal mucosal layer 1201. Therefore, in the reflectance spectra obtained from the normal tissue portion illustrated in FIG. 11A, components derived from the region of the submucosal layer 1202 and the region of the paraffin 1203 are removed by image analyses.

FIG. 11C illustrates first standard deviations σ1 obtained from the normal tissue portion and the abnormal tissue portion. As in the human liver fixed block in the first embodiment, the first standard deviations σ1 tend to be larger in the abnormal tissue portion than in the normal tissue portion.

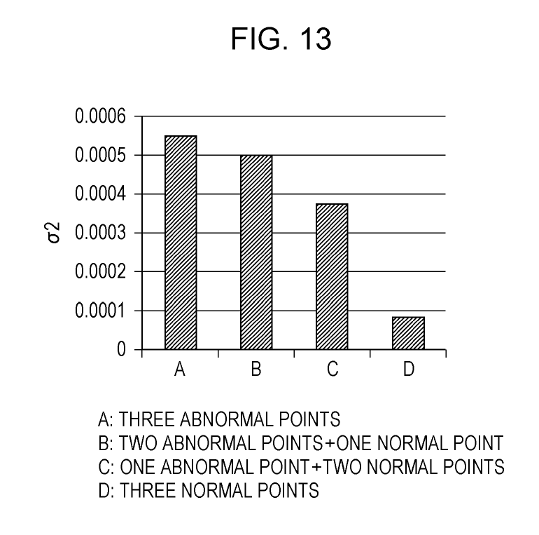

FIG. 13 illustrates second standard deviations σ2 calculated from the first standard deviations σ1 illustrated in FIG. 11C. The second standard deviations σ2 obtained at three measurement points in the normal tissue portion and three measurement points in the abnormal tissue portion are indicated by D and A, respectively. The second standard deviations σ2 obtained from a measurement point group including two measurement points in the abnormal tissue portion and one point in the normal tissue portion and a measurement point group including one measurement point in the abnormal tissue portion and two measurement points in the normal tissue portion are indicated by B and C, respectively. In the case of the human large intestine fixed block, a measurement point group of a second standard deviation σ2 of 0.0001 or smaller can be determined as the normal tissue portion, and a measurement point group of a second standard deviation σ2 more than five times larger than that obtained from the normal tissue portion can be determined as the abnormal tissue portion. A measurement point group of a second standard deviation σ2 between these two ranges can be determined as the boundary portion between the normal tissue portion and the abnormal tissue portion.

That is, according to the apparatus 100 in this example and the determination method using the apparatus 100, the state of the subject can be determined without referring to a database.

Compared to the human liver fixed block in the first example, the first standard deviations σ1 are larger in the human large intestine fixed block in this example. This is because of a difference in tissue structure between the liver, in which homogeneity is high because hepatocytes occupy most of the normal tissue portion, and the large intestine, in which homogeneity is low because the mucosal layer in the normal tissue portion is further divided into an epithelium, a lamina propria, and the like. That is, the first standard deviations σ1 and the second standard deviations σ2, which serve as indices of variation, vary depending on the target organ and the type of cancer.

In the case of the human liver fixed block in the first example, a measurement point group can be determined, even in different subjects, as the normal tissue portion if the second standard deviation σ2 is 0.0002 or smaller or as a hepatoma including the abnormal tissue portion if the second standard deviation σ2 is more than three times larger than that obtained from the normal tissue portion. Therefore, if the target organ and the type of cancer remain the same, the values of indices of variation obtained from another subject can be referred to in a determination of the state of a tissue extracted from a new subject. Since the values of indices of variation vary depending on the target organ and the type of cancer, the states of an organ and a biological tissue can be simultaneously determined by accumulating the values of indices of variation for various organs and cancers, even if the target organ is unknown.

The first and second examples are examples in which the apparatus 100 according to the first embodiment is used. It is possible, however, that when scattering caused by cell clusters is significant in the abnormal tissue portion and accordingly the intensity of a terahertz wave detected is low, the accuracy of obtaining a signal might decrease, thereby making it difficult to calculate variation in spectra. In this case, as in the second embodiment, it is desirable to obtain variation from the amount of change in the percentage contents of the components by modulating the beam diameters of a continuous wave. Variation is obtained by selecting a relatively low frequency side, on which the effect of scattering is small, as the frequencies of the continuous wave and changing the beam diameters as described in the second embodiment.

In a THz-TDS apparatus such as the apparatus 100, the beam diameter and the frequency simultaneously change, and therefore variation in spectra in the abnormal tissue portion can be detected on the basis of both the amount of change in the percentage contents of the components and the sizes of cell clusters in the abnormal tissue portion. If the amount of change in the percentage contents of the components in the abnormal tissue portion is so small that it is difficult to distinguish the change from one in the normal tissue portion or if scattering caused by cell clusters is significant and a high accuracy of obtaining a signal is not ensured, however, use of a continuous wave might be desirable. In the former case, a plurality of continuous waves including different frequency components having the same beam diameter are desirably used, and in the latter case, a plurality of continuous waves having the same frequency and different beam diameters are desirably used.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

For example, although the first standard deviations σ1 and the second standard deviations σ2 are obtained as variation in spectra in the above-described embodiments, the first standard deviations σ1 and the second standard deviations σ2 need not be obtained. It is only necessary that how the optical characteristics change in accordance with the beam diameter or the frequency be identified.

In addition, although variation in spectra is obtained using reflectance spectra as the spectra in the above-described embodiments, the spectra to be used are not limited to these. The state of the subject can be determined using any type of spectra insofar as the spectra are the spectra of the optical characteristics of the subject. For example, the state of the subject can be determined on the basis of variation in spectra by obtaining transmittance spectra or refraction spectra and variation in spectra using the above-described method. The type of spectra is desirably selected on the basis of the type of subject, the state of the subject, the performance of the apparatus, and the like.

This application claims the benefit of Japanese Patent Application No. 2013-242004, filed Nov. 22, 2013 and Japanese Unexamined Patent Application Publication No. 2014-211197, filed Oct. 15, 2014, which are hereby incorporated by reference herein in their entirety.

What is claimed is:

1. An information obtaining apparatus that obtains information regarding a subject, the information obtaining apparatus comprising:
a radiation unit configured to focus a terahertz wave having a plurality of different frequencies or a plurality of different beam diameters and radiate the terahertz wave onto the subject;
a detection unit configured to detect the terahertz wave that has penetrated the subject or that has been reflected by the subject;
a spectrum obtaining unit configured to obtain a spectrum of optical characteristics of the subject using a result of the detection performed by the detection unit; and
a determination unit configured to determine a state of the subject on the basis of the spectrum obtained by the spectrum obtaining unit,
wherein the determination unit determines the state of the subject on the basis of a difference between a plurality of spectra obtained by the spectrum obtaining unit at a plurality of different positions in the subject.

2. The information obtaining apparatus according to claim 1,
wherein the difference is a difference between a plurality of spectra indicating changes in the optical characteristics of the subject in accordance with the frequencies or the beam diameters of the terahertz wave in each of the plurality of spectra.

3. The information obtaining apparatus according to claim 1,
wherein the beam diameters of the terahertz wave when the terahertz wave is radiated onto the subject are 0.5 to 3.0 millimeters.

4. The information obtaining apparatus according to claim 1,
wherein the terahertz wave is a pulsed wave having a plurality of beam diameters that are different in accordance with a plurality of different frequencies, and
wherein the spectrum obtaining unit obtains the spectrum using a time waveform obtained from the result of the detection performed by the detection unit.

5. The information obtaining apparatus according to claim 1,
wherein the radiation unit focuses the terahertz wave such that centers of the beam diameters of the terahertz wave match and then radiates the terahertz wave onto the subject.

6. The information obtaining apparatus according to claim 1, further comprising:
an image forming unit configured to form an image of the subject,
wherein the image forming unit forms the image of the subject on the basis of the state of the subject determined by the determination unit.

7. A method for obtaining information regarding a subject, the method comprising the steps of:
obtaining a spectrum of optical characteristics of the subject using a result of detection of a terahertz wave having a plurality of different frequencies or a plurality of different beam diameters that has been focused, that has been radiated onto the subject, and that has penetrated the subject or that has been reflected by the subject; and
determining a state of the subject on the basis of the spectrum obtained in the step of obtaining,
wherein, in the step of determining, the state of the subject is determined on the basis of a difference between a plurality of spectra obtained at a plurality of different positions in the subject.

8. The method according to claim 7,
wherein, in the step of determining, the state of the subject is determined by comparing the difference, a difference between a plurality of spectra in a first region of the subject obtained in advance, a difference between a plurality of spectra in a second region, whose state is different from a state of the first region, obtained in advance, and a difference between a plurality of spectra in a boundary region between the first region and the second region obtained in advance.

9. The method according to claim 8, further comprising the steps of:
arbitrarily selecting a measurement point group including a plurality of different positions in the subject; and
obtaining a difference between a plurality of spectra obtained by radiating the terahertz wave onto each of the positions included in the measurement point group,
wherein, by repeating the step of arbitrarily selecting and the step of obtaining a difference, the difference in the first region, the difference in the second region, and the difference in the boundary region between the first region and the second region are obtained.

10. The method according to claim 8,
wherein, in the step of determining, a standard deviation is obtained on the basis of values of the optical characteristics of the plurality of spectra at each of the frequencies or the beam diameters of the terahertz wave, and
wherein the state of the subject is determined by comparing the standard deviation, a standard deviation in the first region obtained in advance, a standard deviation in the second region obtained in advance, and a standard deviation in the boundary region between the first region and the second region obtained in advance at each of the plurality of different frequencies or beam diameters.

11. The method according to claim 8,
wherein, in the step of determining, a second standard deviation is obtained from a first standard deviation obtained on the basis of the values of the optical characteristics of the plurality of spectra at each of the frequencies or the beam diameters of the terahertz wave, and
wherein the state of the subject is determined by comparing the second standard deviation, the standard deviation in the first region obtained in advance, the standard deviation in the second region obtained in advance, and the standard deviation in the boundary region between the first region and the second region obtained in advance.

12. The method according to claim 7,
wherein the subject is a biological tissue, and
wherein, in the step of determining, a normal tissue portion, an abnormal tissue portion, and a boundary portion between the normal tissue portion and the abnormal tissue portion are distinguished in the biological tissue as the state of the subject.

13. The method according to claim 7, further comprising the step of:
forming an image of the subject on the basis of a result of the determination made in the step of determining.

14. A computer-readable recording medium storing a program for causing a computer to execute each of the steps included in the method according to claim 7.
